(19) United States
(12) Patent Application Publication
(10) Pub. No.: US 2009/0191628 A1
(43) Pub. Date: Jul. 30, 2009

(54) Methods for co-culturing cord blood derived cells with menstrual stem cells

(75) Inventors: Mercedes A. Walton, Mendham, NJ (US); Julie G. Allickson, Odessa, FL (US)

Correspondence Address: DILWORTH PAXSON LLP, 1500 Market Street, Suite 3500 E, PHILADELPHIA, PA, 19102, US

(73) Assignee: Cryo-Cell International, Inc., Oldsmar, FL (US)

(21) Appl. No.: 12/290,551

(22) Filed: Oct. 31, 2008

Related U.S. Application Data
(60) Provisional application No. 61/001,456, filed on Oct. 31, 2007.

(51) Int. Cl. C12N 5/08

(52) U.S. Cl. 435/372

(57) ABSTRACT

Methods are provided for obtaining expanded human cord blood cells expressing CD34. The methods involve seeding a sufficient amount of cord blood cells with a sufficient amount of menstrual cells under co-culture conditions suitable to promote expansion of the cord blood cells, and co-culturing the cord blood cells with the menstrual cells under culture conditions that support at least two or more population doublings of the cord blood cells. Methods are also provided for growing expanded human cord blood cells to give rise to any one of colony forming units, colony forming unit granulocyte macrophages (CFU-GM), burst forming unit erythroids (BFU-E), and colony forming unit granulocyte erythrocyte macrophage megakaryocyte (CFU-GEMM) blood lineage precursor cells. The expanded cells may express CD34, SSEA-4, and HLA-II. Compositions of the expanded cells are also provided.

### CROSS-REFERENCE TO RELATED APPLICATIONS

This application claims the priority of U.S. Provisional Patent Application Ser. No. 61/001,456, filed Oct. 31, 2007, entitled “Methods for Co-Culturing Cord Blood Derived Cells with Menstrual Stem Cells” the entirety of which is incorporated herein by reference.

### FIELD OF THE INVENTION

The invention relates generally to human cell culture and methods for enhancing isolated cell populations through co-culture. More specifically, the invention relates to the co-culturing cord blood derived cells with menstrual stem cells so as to obtain improved cell populations of expanded cord blood derived cells expressing CD34.

### BACKGROUND OF THE INVENTION

Umbilical cord blood is a recognized source of stem cells with the capability to treat a number of disorders of the human body. Cord blood is a rich source of hematopoietic progenitor cells including mononuclear cells containing CD34 cells. Families may decide to collect umbilical cord blood because of the potential benefits of having a rich source of stem cells stored in case of a medical need of the child or family member.

Cord blood, also referred to as umbilical cord blood, is blood remaining in the umbilical cord and placenta at the time of birth. This blood is a rich source of stem cells, which can be collected, processed, and cryogenically preserved for potential, future use. The cord blood stem cells are beneficial because they have a high rate of engraftment, are more tolerant of tissue mismatches, have a lower rate of severe graft-vs-host disease (a major complication in stem cell transplants), and are rarely contaminated with latent viruses.

The number of human deficiencies treatable with cord blood has increased significantly over the past decade. By way of example, cord blood cells have been used to treat at least 70 forms of various forms of cancers, syndromes associated with bone marrow failures, blood disorders, metabolic disorders, immunodeficiency disorders, and other disease states. For example, cord blood cells have been used to treat the following: Acute lymphoblastic leukemia (ALL), Acute myeloid leukemia (AML), Burkitt's lymphoma, Chronic myeloid leukemia (CML), Juvenile myelomonocytic leukemia (JMML), Hemophagocytic lymphohistiocytosis, Non-Hodgkin's lymphoma, Hodgkin's lymphoma, Langerhan's cell histiocytosis, Lymphomatoid granulomatosis, Myelodysplastic syndrome (MDS), Chronic myelomonocytic leukemia (CMML), Amegarakyocytic thrombocytopenia, Autoimmune neutropenia (severe), Congenital dyserythropoietic anemia, Cyclic neutropenia, Diamond-Blackfan anemia, Evan's syndrome, Fanconi anemia, Glanzmann's disease, Juvenile dermatomyositis, Kostmann's syndrome, Red cell aplasia, Schwachman syndrome, Severe aplastic anemia, Congenital sideroblastic anemia, Thrombocytopenia with absent radius (TAR syndrome), Dyskeratosis congenital, Sickle-cell anemia (hemoglobin SS), HbSC disease, Sickle β-Thalassemia, α-thalassemia major (hydrops fetalis), β-thalassemia major (Cooley's anemia), β-thalassemia intermedia, E-β thalassemia, E-β+ thalassemia, Adrenoleukodystrophy, Gaucher's disease (infantile), Metachromatic leukodystrophy, Krabbe disease (globoid cell leukodystrophy), Gunther disease, Hermansky-Pudlak syndrome, Hurler syndrome, Hurler-Scheie syndrome, Hunter syndrome, Sanfilippo syndrome, Maroteaux-Lamy syndrome, Mucolipidosis Type II, III, Alpha mannosidosis, Neumann Pick Syndrome, type A and B, Sandoff Syndrome, Tay Sachs Disease, Batten disease (inherited neuironal ceroid lipofuscinosis), Lesch-Nyhan disease, Ataxia telangectasia, Chronic granulomatous disease, DiGeorge syndrome, IKK gamma deficiency, Immune dysregulation polyendocrineopathy X-linked, Mucolipidosis, Type II, Myelokathesis, X-linked immunodeficiency, Severe combined immunodeficiency, Adenosine desaminase deficiency, Wiskott-Aldrich syndrome, X-linked agammaglobulinemia, X-linked lymphoproliferative disease, Omenn's syndrome, Reticular dysplasia, Thymic dysplasia, Leukocyte adhesion deficiency, and Osteopetrosis.

There are limitations with cord blood cell collection and use in treating human disorders. First, cord blood cells may only be collected immediately after birth. This places significant limitations on the number of times that umbilical cord blood may be collected. Second, umbilical cord blood is collected in a small volume containing a limited amount of stem cells. Certain disorders require infusion or transplantation of a large number of stem cells. The small amount of collectable cord blood cells makes use of such cells for therapies requiring a large number of cells infeasible.

Research is ongoing to develop advancements to overcome the limitations of cord blood cells. Techniques have been developed to combine multiple cord blood units or to expand stem cells in a single cord blood unit prior to transplantation. Such techniques were developed to address the problem of having too little a population of stem cells to treat a disorder. Even with the developments, umbilical cord blood stem cells prove to be difficult to expand in cell culture. The rich, but limited, source of stem cells still has restrictions for treatment of disorders.

In vitro assay systems have been developed to quantify multi-potential progenitors and lineage-restricted progenitors of the erythrocyte, granulocyte, monocyte-macrophage, and megakaryocyte myeloid cell lineages. When cultured in a suitable semi-solid matrix, individual progenitors called colony-forming cells (CFCs) proliferate to form discrete cell clusters or colonies. CFC assays are performed by placing a cell suspension into a semi-solid medium, such as methylcellulose or collagen supplemented with nutrients and cytokines, followed by incubation. The CFCs are then classified and enumerated based on the morphological recognition of one or more types of hematopoietic lineage cells within the colony. Colony evaluation and enumeration can be done in situ by light microscopy or by plucking individual colonies and then staining the cells using cytochemical and immunocytochemical methods.

Various gelling agents including methylcellulose have been used for CFC assays. Methylcellulose is a relatively inert polymer that forms a stable gel with good optical clarity. It is commonly used in culture medium supplemented with compounds including fetal bovine serum (FBS), bovine serum albumin (BSA), 2-mercaptoethanol, insulin, transferrin, and recombinant cytokines or conditioned medium as a source of colony-stimulating factors. Methylcellulose-based medium permits better growth of erythroid lineage cells than other types of semi-solid matrices, thus allowing the assay of erythroid, granulocyte, monocyte and multi-potential CFCs within the same culture. This media allows for the detection and enumeration of human colony-forming unit-erythroid (CFU-E), burst-forming unit-erythroid (BFU-E), CFU-granulocyte macrophage (CFU-GM) and CFU-granulocyte, erythrocyte, macrophage, megakaryocyte (CFU-GEMM).

Although advances have been made, there remains a need for methods to improve the expansion of umbilical cord stem cells to produce a larger quantity of stem cells for use in treating human disorders. The present invention is directed toward meeting this need.

### SUMMARY OF THE INVENTION

The invention is based on the discovery that stem cells from umbilical cord blood proliferate in sufficiently greater numbers when co-cultured with a population of menstrual cells. The discovery has shown that the menstrual stem cells provide a supporting function in culture with cord blood stem cells to enhance proliferation of the cord blood cells. The cord blood cells are collected according to current industry standards for collection, cryopreservation, and storage. The menstrual stem cells used for co-culturing with cord blood stem cells may be collected according to the teachings of U.S. Patent Publication No. 20080241113. Co-culturing of cord blood cells and menstrual cells creates a culture environment that promotes the expansion of cells expressing CD34, SSEA4, and HLA-II.

The teachings of U.S. Patent Publication No. 20080241113 provide a number of methods for collecting menstrual stem cell populations that are suitable for use in the invention. The menstrual stem cells that are used for co-culturing may be obtained from fresh or cryopreserved menstrual stem cells. The menstrual stem cells may be isolated for CD117 or other cell surface marker(s) and may also be expanded through cell culture. The menstrual stem cells may also be isolated for certain cell markers and then cultured for expansion. Any population of menstrual stem cells described in U.S. Patent Publication No. 20080241113 may be used in the co-culturing methods and processes of the invention.

Accordingly, the invention provides methods for co-culturing cord blood cells with menstrual cells for improved proliferation of the cord blood cells.

In a related aspect the invention provides a population of human cells expressing CD34 obtained from expansion of human cord blood cells in suitable culture conditions with human menstrual cells to promote population doublings of the human cord blood cells. The population of cells express SSEA4 and HLA-II.

The population of cells of the invention may be suspended in any one of a cryopreservation agent, a culture media, a growth media, or a differentiation media.

The population of human cells expressing CD34 of the invention result from at least two of more population doublings.

The population of cells of the invention are capable of giving rise to any one of colony forming units, colony forming unit granulocyte (CFU-GM) macrophages, burst forming unit erythiroids (BFU-E), and colony forming unit granulocyte erythrocyte macrophage megakaryocyte (CFU-GEMM) blood lineage precursor cells.

In another aspect, the invention also provides a population of human cord blood cells expressing CD34 obtained by the process comprising co-culturing a sufficient amount of cord blood stem cells with a sufficient amount of menstrual stem cells in conditions suitable for expansion of the cord blood cells, and then proliferating the sufficient amount of cord blood cells in culture through at least two population doublings. The step of proliferating the sufficient amount of cord blood cells in culture comprises growing the cord blood cells to give rise to any one of colony forming units, colony forming unit granulocyte macrophages (CFU-GM), burst forming unit erythroids (BFU-E), and colony forming unit granulocyte erythrocyte macrophage megakaryocyte blood lineage precursor cells (CFU-GEMM).

In an embodiment, the process of the invention may comprise a step of growing the sufficient amount of cord blood cells in culture to give rise to any one of colony forming units (CFU), colony forming unit granulocyte macrophages (CFU-GM), burst forming unit erythroids (BFU-E), and colony forming unit granulocyte erythrocyte macrophage megakaryocyte blood lineage precursor cells (CFU-GEMM).

In yet another embodiment, the process of the invention may comprise a step of isolating cord blood cells expressing CD34 after proliferating the sufficient amount of cord blood cells in culture.

In a further embodiment, the process of the invention may comprise a step of cryo-preserving the population of human cord blood cells expressing CD34 after proliferating the sufficient amount of cord blood cells in culture.

The population of human cord blood cells expressing CD34 obtained by the process of the invention may also express at least one of CD34, SSEA4, and HLA-II after proliferating the sufficient amount of cord blood cells under suitable culture conditions.

In yet a further aspect, the invention provides a method for obtaining expanded human cord blood cells expressing CD34. The method of the invention comprises the steps of seeding a sufficient amount of cord blood cells with a sufficient amount of menstrual cells under co-culture conditions suitable to promote expansion of the cord blood cells, and co-culturing the cord blood cells with the menstrual cells under culture conditions that support at least two or more population doublings of the cord blood cells.

In an embodiment, the co-culturing of the human cord blood cells expressing CD34 comprises expanding cord blood cells to express at least one or more of SSEA4 and HLA-II.

In another embodiment, the expanded human cord blood cells of the invention express high levels of CD34. Furthermore, the co-culturing of the cord blood cells of the invention comprises expanding cord blood cells to give rise to any one of colony forming units, colony forming unit granulocyte macrophages (CFU-GM), burst forming unit erythroids (BFU-E), and colony forming unit granulocyte erythrocyte macrophage megakaryocyte blood lineage precursor cells (CFU-GEMM).

In alternative embodiments, the methods of the invention may comprise at least one of the further steps of immunoselecting expanded human cord blood cells for CD34, isolating expanded human cord blood cells from a culture for infusion into a human, cryopreserving expanded human cord blood cells, or differentiating expanded cord blood cells into a cell lineage.

In yet another embodiment, the methods of the invention comprise the step of growing expanded human cord blood cells to give rise to any one of colony forming units, colony forming unit granulocyte macrophages (CFU-GM), burst forming unit erythroids (BFU-E), and colony forming unit granulocyte erythrocyte macrophage megakaryocyte (CFU-GEMM) blood lineage precursor cells.

### BRIEF DESCRIPTION OF THE DRAWINGS

FIG. 1 shows a schematic flow chart of the present invention.

FIGS. 2a and 2b are photographs of cell culture of Cord 871R cells plated after thaw and cultured in Methocult 4053 semi-solid methylcellulose media for hematopoietic colony forming cells described in Example 1. Cord 871R cells were plated at 50,000 cells per well in one ml of media per well. FIG. 2a is a photograph (200×) of cells after 8 days of cell culture.

FIG. 2b is a photograph (40×) of cells after 9 days of cell culture. FIGS. 2a and 2b demonstrate cell free in semi-solid culture media without growth of colony forming units (CFU).

FIGS. 3a-3l are photographs of M28100RM menstrual cells in culture in methylcellose media for CFUs described in Example 2. FIG. 3a is a photograph (100×) of the cells in culture showing a BFU-E which demonstrates a red hue from the hemoglobin production after plating 10,000 cells per well and 8 days of cell culture. FIG. 3b is a photograph (100×) of free cells in the methylcellose media for colony forming units after 8 days of culture. FIGS. 3c and 3d are photographs (200×) of cells in culture showing a BFU-E which demonstrates a red hue from the hemoglobin production after plating cells at 21,600 cells per well and 8 days in cell culture. FIG. 3e is a photograph (40×) of cells in culture showing initial BFU-E production after plating cells at 5,000 cells per well and 8 days of culture. FIGS. 3f, 3g, and 3h are photographs (100×, 100×, and 40×, respectively) of cells in culture showing a BFU-E which demonstrates a red hue from the hemoglobin production after plating 10,000 cells per well and 9 days of cell culture. FIGS. 3i, 3j, and 3k are photographs (100×, 100×, and 40×, respectively) of cell culture showing BFU-E which demonstrates a red hue from the hemoglobin production after plating 21,600 cells per well and 9 days of culture. FIG. 3l is a photograph of free cells in the media without colony forming potential at this concentration.

FIGS. 4a-4d are photographs of M28101R menstrual cells plated at 5,000, 10,000, and 21,600 cells per well. FIG. 4a is a photograph (40×) of the cells in culture showing a CFU-GM after plating M28101R menstrual cells at 10,000 cells per well and 8 days of cell culture. FIG. 4b is a photograph (40×) of the cells in culture showing CFU-GM production after plating cells at 10,000 cells per well and 9 days of culture. FIG. 4c is a photograph (100×) of the cells in culture showing BFU-E production after plating cells at 21,600 cells per well and days of cell culture. FIG. 4d is a photograph (40×) of free cells after plating cells at 5,000 cells per well and 9 days of cell culture.

FIGS. 5a-5g are photographs of co-culture of M28100RM menstrual cells and Cord 871R cells described in Example 4. FIG. 5d is a photograph (40×) of part of a CFU-GM with menstrual cells plated at 5,000 cells per well and cord blood cells plated at 50,000 cells per well after 9 days of culture. FIGS. 5a, 5e and 5g show a photograph (100×) of M28100RM menstrual cells plated at 10,000 cells per well and Cord 871R cells plated at 50,000 cells per well after 8 days of culture (FIGS. 5a and 5e) and 9 days of culture (FIG. 5g). FIGS. 5a and 5e demonstrate CFU-GM colony formation. FIG. 5g demonstrates BFU-E colony formation. FIGS. 5b, 5c, and 5f are photographs (200×, 100×, and 100×, respectively) of M28100RM menstrual cells plated at 21,600 cells per well and Cord 871R cells plated at 50,000 cells per well after 8 days of cell culture (FIGS. 5b and 5c) and 9 days of culture (FIG. 5f).

FIGS. 6a-6e are photographs of co-culture of M2801R menstrual cells and Cord 871R cells described in Example 5. FIGS. 6a, 6b, and 6c demonstrate free cells in the methylcellose semi-solid hematopoietic media. M28101R menstrual cells cultured alone were able to produce CFU-GM and BFU-E when plated at different concentrations.

### DESCRIPTION OF THE EMBODIMENTS OF THE INVENTION

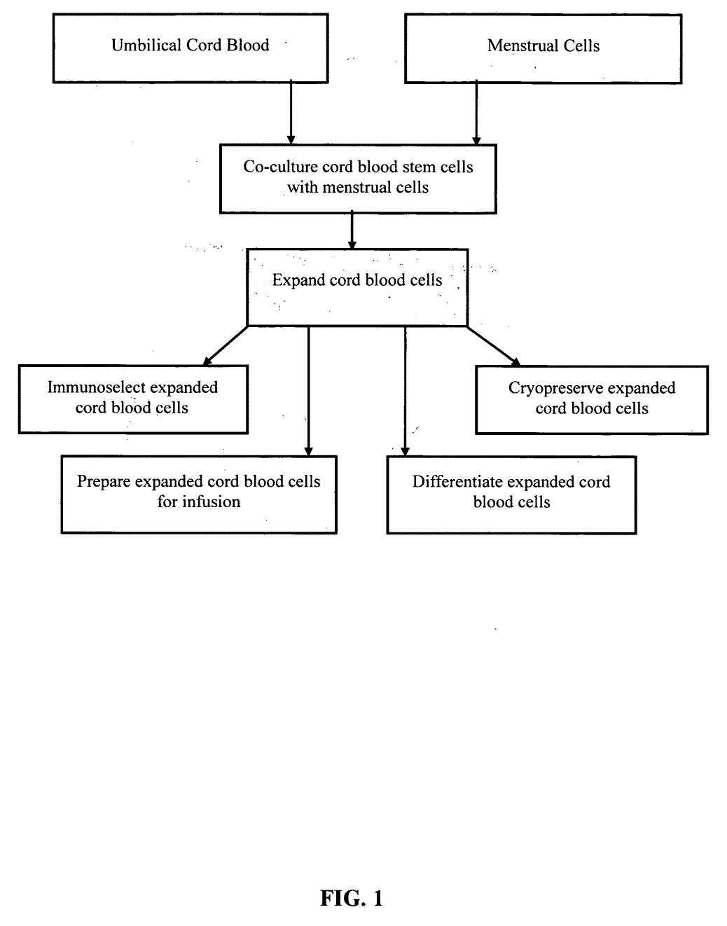

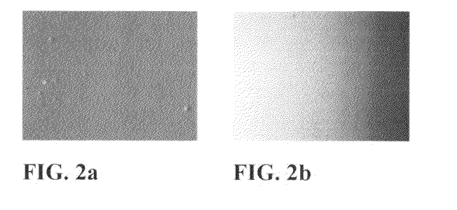

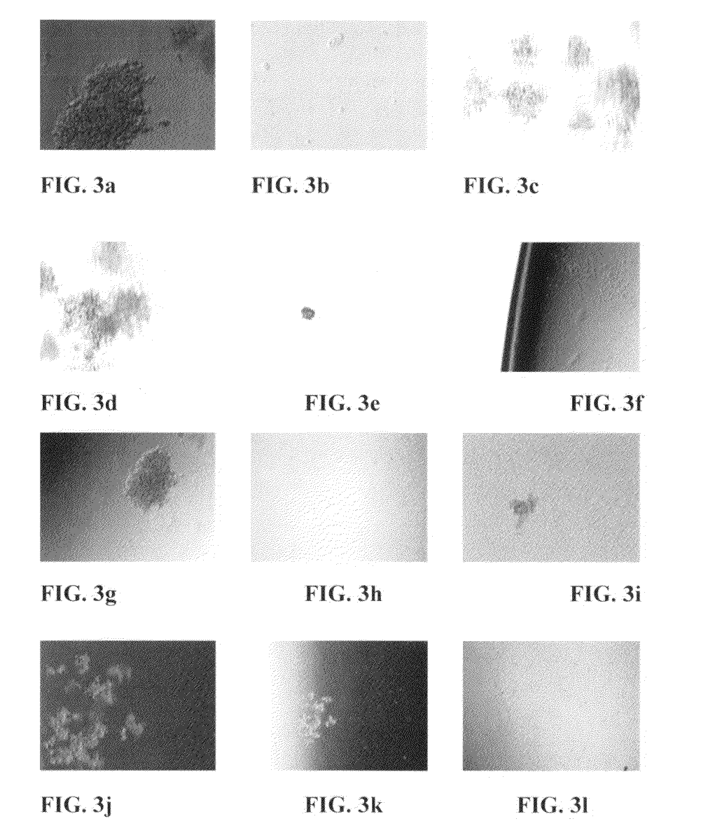

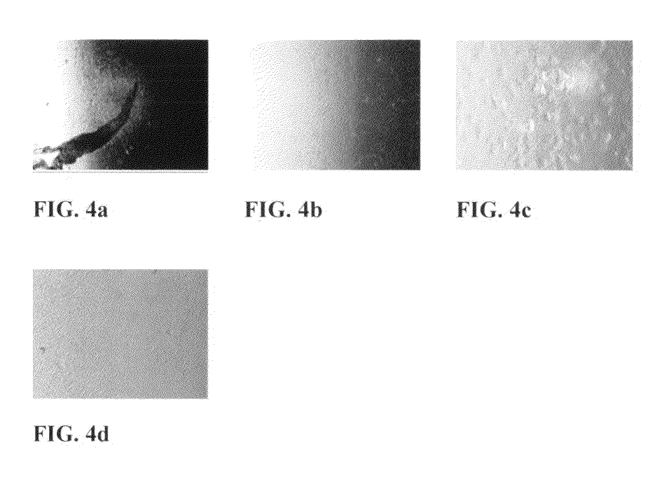

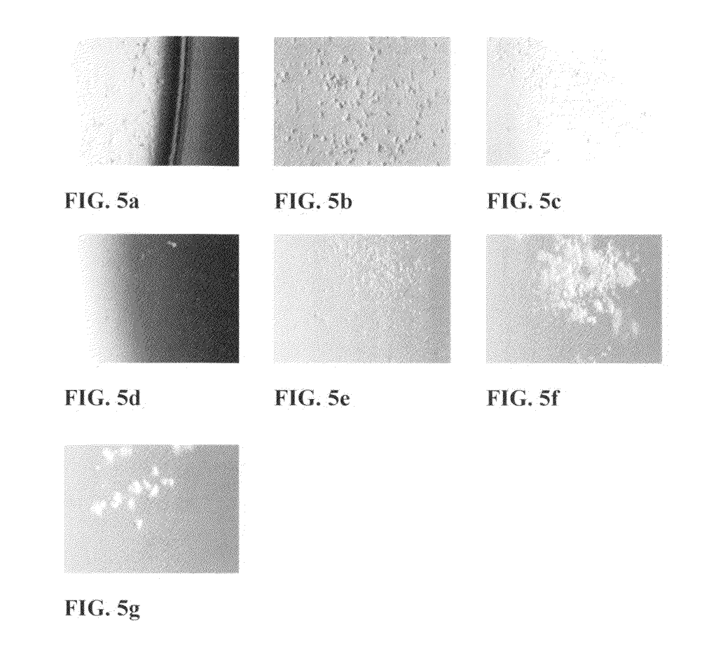

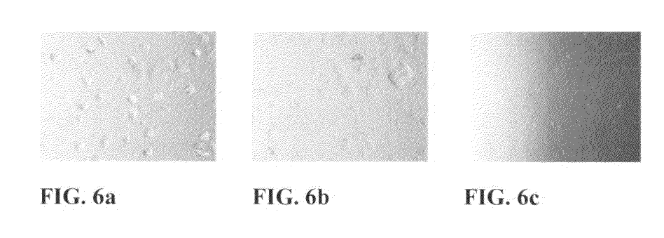

In reference to FIGS. 1-6, processes and methods for co-culturing umbilical cord blood cells with menstrual cells to expand the number of cells expressing CD34 are provided by present invention. Compositions of expanded human cells expressing CD34 obtained by the processes and methods of the invention are also provided.

Whole cord blood is a rich source of hematopoietic progenitor cells including mononuclear cells containing CD34+ cells. Cord blood stem cells comprise mononuclear cells including CD34+ cells. Cord blood stem cells are obtained from whole cord blood extracted from the umbilical immediately after deliver of a baby, but generally before the placenta has been delivered. The moment of delivery is the only opportunity to harvest a newborn's stem cells. And collection is safe for both vaginal and cesarean deliveries. To collect cord blood, cord blood is drawn from the umbilical cord into a blood collection bag. The cord blood is packaged in shipping materials and shipped to a laboratory for processing within 36 to 48 hours of collection.

Whole blood is collected from the umbilical cord following the birth of a baby after clamping the cord. The volume of the whole cord blood may be about 110 ml. The collected cord blood sample is shipped under industry standards to a laboratory for processing.

Cord blood is processed under aseptic conditions using density gradient separation (Ficoll/Hypaque) to separate mononuclear cells containing a population of cells expressing CD34. Cord blood is aliquoted into sterile 50 ml tubes. The tubes are centrifuged for about 15 minutes at 470 g. After centrifugation, the packed cells should be at a 1:3 ratio per volume. Plasma may be removed from the tube after centrifugation, or DPBS may be added to the tube, to achieve the 1:3 ratio of packed cells to volume. Each tube should have no more than about 35 ml volume. Underlay each tube with 10 ml of LSM. Centrifuge each tube for about 30 minutes at 400 g. Remove the top layer of plasma. Remove the next layer containing mononuclear cells and plasma and transfer it to another 50 ml tube.

The cells in the 50 ml tube should be suspended up to a volume of about 45 ml using RPM101640 1× with L-glutamine at about a 1:2 ratio of RPMI to cell mixture. The tube with cellular suspension may be centrifuged for about 15 minutes at 470 g. After centrifugation, the supernatant is removed. Add a small amount of RPMI to resuspend the pellet. Transfer the cellular suspension to a 15 ml tube. Centrifuge the cellular suspension in the 15 ml tube for about 15 minutes at 400 g. Decant the supernatant and resuspend the pellet up to 5 ml using RPMI. Add 5 ml of a DMSO/autologous plasma (1 ml DMSO and 4 ml of autologous plasma) dropwise. Reduce the temperature of the cellular suspension in DMSO/autologous plasma in a control rate freezer to about −85° C. Place the tubes in a liquid nitrogen tank at about −185° C. or less.

The phrase “menstrual cells” is used in reference to cells collected from menstrual flow according any of the methods of U.S. Patent Publication No. 20080241113. The menstrual cells comprises cells expressing at least one of the cell markers or intracellular markers including, but not limited to, CD9, CD10, CD13, CD29, CD44, CD49e, CD49f, CD59, CD81, CD105, CD166, and HLA class I, while expressing low or no levels of CD3 and MHC II. While the aforementioned characteristics of the cell are provided as exemplary characteristics, additional and alternative cell surface characteristics of menstrual cells are provided throughout the disclosure in U.S. Patent Publication No. 20080241113, including, but not limited to, the characteristics provided on any table and figures thereto before and after cryopreservation, before and after CD117 selection, before and after cell culture, or any combination disclosed in U.S. Patent Publication No. 20080241113. Moreover, U.S. Patent Publication No. 20080241113 is incorporated herein by reference in its entirety, and provides further disclosure regarding the menstrual cells of the invention. The menstrual cells used for co-culturing with cord blood stem cells may be collected from menstrual flow, concentrated, and cryopreserved according to the teachings of U.S. Patent Publication No. 20080241113 and later thawed according to the methods of the invention. Alternatively, the teachings of U.S. Patent Publication No. 20080241113 provide a number of methods for obtaining menstrual cells that are suitable for use in the invention.

While the term “cell” may be used in the singular sense in the application, the term “cells” may also be used to refer to more than one cell used with the invention.

It is recognized that certain cells are pluripotent in nature due to the capability of the cells to differentiate into a number of distinct cell types. A pluripotent cell possesses the capacity to undergo differentiation into a number of different mammalian cell types. By way of example, the menstrual cells used in the invention show potential to differentiate into various cell lineages, such as, for example, neural, cardiogenic, chondrogenic, adipogenic, and osteogenic lineages.

Methods and Compositions of the Present Invention

The use of the expanded CD34 cells for therapeutic use may be beneficial for a number of reasons. In particular, the expanded CD34 cells (a) require the use of less cord blood cells for proliferation in comparison to other cord blood cell expansion methods, (b) are proliferative in co-cuilture with menstrual cells, (c) are capable of autologous applications, (d) are capable of allogenic applications, and (e) may be used as a source for customized regenerative healthcare solutions.

The invention provides a population of human cells expressing CD34 obtained from expansion of human cord blood cells in suitable culture conditions with human menstrual cells to promote population doublings of the human cord blood cells. The population of cells also express SSEA4 and HLA-II.

The population of cells of the invention may be suspended in any one of a cryopreservation agent, a culture media, a growth media, or a differentiation media.

The population of human cells expressing CD34 of the invention result from at least two of more population doublings.

The population of cells of the invention are capable of giving rise to any one of colony forming units, colony forming unit granulocyte (CFU-GM) macrophages, burst forming unit erythroids (BFU-E), and colony forming unit granulocyte erythrocyte macrophage megakaryocyte (CFU-GEMM) blood lineage precursor cells.

The invention also provides a population of human cord blood cells expressing CD34 obtained by a process comprising co-culturing a sufficient amount of cord blood stem cells with a sufficient amount of menstrual cells in conditions suitable for expansion of the cord blood cells, and then proliferating the sufficient amount of cord blood cells in culture through at least two population doublings. The step of proliferating the sufficient amount of cord blood cells in culture comprises growing the cord blood cells to give rise to any one of colony forming units, colony forming unit granulocyte macrophages (CFU-GM), burst forming unit erythroids (BFU-E), and colony forming unit granulocyte erythrocyte macrophage megakaryocyte blood lineage precursor cells (CFU-GEMM).

The process of the invention may comprise a step of growing the sufficient amount of cord blood cells in culture to give rise to any one of colony forming units (CFU), colony forming unit granulocyte macrophages (CFU-GM), burst forming unit erythroids, (BFU-E), and colony forming unit granulocyte erythrocyte macrophage megakaryocyte blood lineage precursor cells (CFU-GEMM).

The process of the invention may comprise a step of isolating cord blood cells expressing CD34 after proliferating the sufficient amount of cord blood cells in culture.

The process of the invention may comprise a step of cryopreserving the population of human cord blood cells expressing CD34 after proliferating the sufficient amount of cord blood cells in culture.

The population of human cord blood cells expressing CD34 obtained by the process of the invention may also express at least one of CD34, SSEA4, and HLA-II after proliferating the sufficient amount of cord blood cells under suitable culture conditions.

The invention provides a method for obtaining expanded human cord blood cells expressing CD34. The method of the invention comprises the steps of seeding a sufficient amount of cord blood cells with a sufficient amount of menstrual cells under co-culture conditions suitable to promote expansion of the cord blood cells, and co-culturing the cord blood cells with the menstrual cells under culture conditions that support at least two or more population doublings of the cord blood cells.

The co-culturing of the human cord blood cells expressing CD34 comprises expanding cord blood cells to express at least one or more of SSEA4 and HLA-II. The expanded human cord blood cells of the invention express high levels of CD34.

The co-culturing of the cord blood cells of the invention comprises expanding cord blood cells to give rise to any one of colony forming units, colony forming unit granulocyte macrophages (CFU-GM), burst forming unit erythroids (BFU-E), and colony forming unit granulocyte erythrocyte macrophage megakaryocyte blood lineage precursor cells (CFU-GEMM).

The methods of the invention may comprise at least one of the further steps of immunoselecting expanded human cord blood cells for CD34, isolating expanded human cord blood cells from a culture for infusion into a human, cryopreserving expanded human cord blood cells, or differentiating expanded cord blood cells into a cell lineage.

The methods of the invention comprise the step of growing expanded human cord blood cells to give rise to any one of colony forming units, colony forming unit granulocyte macrophages (CFU-GM), burst forming unit erythroids (BFU-E), and colony forming unit granulocyte erythrocyte macrophage megakaryocyte (CFU-GEMM) blood lineage precursor cells.

Preparation of Cells for Culture

The cord blood cell sample and the menstrual cell sample may be cryopreserved in storage. Alternatively, either or both of the cord blood cell sample and the menstrual cell sample may be fresh after being processed from collected raw blood samples. In cases where either or both of the cord blood cell sample and the menstrual cell sample are cryopreserved, the cryopreserved cord blood cells and/or menstrual cells must be thawed in preparation for culture. In cases where either or both of the cord blood cell sample and the menstrual cell sample are fresh, the cells may be prepared for culture.

The process for thawing cryopreserved cells comprises steps of thawing cryopreserved cells and then washing them through centrifugation. Cryopreserved cells are thawed by removing a vial of cells from cryopreservation and agitating the vial in an about 37-40° C. water bath until a few pieces of frozen sample remain. The cryopreserved cells should not thaw completely. The partially thawed cells are transferred to chilled Chang's complete media with DNase (10 drops per 100 ml) and mixed gently by inversion. Chang's complete media should be mixed at a 5:1 ratio to the partially thawed cells. For example, 25 ml of Chang's complete media is combined with 5 ml of thawing cells. At this point, an about 100-200 ul sample of Chang's complete media and thawing cells may be removed for flow cytometry analysis described in further detail below.

The solution of Chang's complete media suspending thawing cells may then be subjected to a first step of centrifugation at about 120 g for about 5 minutes at about ambient temperature. Once centrifugation is complete, the supernatant is removed and the pellet of cells and possibly other debris is resuspended in Chang's complete media without DNase by gentle inversion. The cells suspended in Chang's complete media may then be subjected to a second step of centrifugation at about 120 g for about 5 minutes at about ambient temperature. Once the second centrifugation step is complete, the supernatant is removed and the pellet of cells is resuspended in 7 ml of 15% FBS Chang's growth media.

Chang's complete media comprises MEM alpha media, Chang B, Chang C, Penicillin/Streptomycin, L-glutamine, and ES-FBS. Chang's complete media is prepared by combining 650 ml of MEM alpha media, 180 ml of Chang B (basal media) (18% v/v), 20 ml of Chang C (2% v/v), 10 ml of Penicillin/Streptomycin (10,000 units/ml Penicillin G Sodium and 10,000 ug/ml Streptomycin sulfate), 10 ml of L-glutamine 200 mM (100×), and 150 ml of ES-FBS (19% v/v).

The process of thawing and washing cryopreserved cells may be used to prepare cryopreserved umbilical cord blood cells and menstrual cells for culture.

Expansion of Cells through Culture in Flasks

Thawed or fresh cord blood cells are plated with thawed or fresh menstrual cells to co-culture the cells in a flask. Cord blood cells and menstrual cells may be co-cultured in T-25 non-tissue culture treated flasks. Cells should not exceed about 10,000,000 cells per flask. A sufficient number of umbilical cord blood cells are co-cultured with a sufficient number of menstrual cells in a flask. In an embodiment, the cord blood cells may range from about 1,000 cells to about 10,000 cells while the menstrual cells may range from about 10,000 cells to about 50,000 cells. Other amounts of umbilical cord blood cells and menstrual cells may be sufficient so long as the menstrual cells provide a support function to promote the expansion of the cord blood cells.

Previously cultured cord blood cells and menstrual cells may be plated at about 2,000 per cm² with about a 48 hour passage time. If more cells are plated, the cells may go through passages in about 24 hours. The cord blood cells and menstrual cells may be plated in T-25, T-75, and T-175 non-tissue culture treated flasks with about 7 ml, about 15 ml, and about 30 ml of Chang's complete media, respectively.

The co-culture of cord blood cells and menstrual cells may be incubated in a CO₂ incubator at about 36° C. to about 38° C. until the cells are confluent at about 70-80%.

The co-culture of cord blood cells and menstrual cells may be dissociated from the flask by a trypsinizing step. When it is apparent that the co-cultured cord blood and menstrual cells are ready for dissociation, the media in the flask should be aspirated and then the non-tissue culture flask is washed with DPBS without calcium or magnesium in a volume of about 5 ml for a T-25 flask, about 10 ml for a T-75 flask, or about 25 ml for a T-157 flask. After the washing, the cells may be coated with TrypLE enzyme at about 36° C. to about 38° C. at a volume of about 1.5 ml for a T-25 flask, about 3 ml for a T-75 flask, and about 6 ml for a 175 flask. The TrypLE enzyme may be incubated with the cells for about 5 minutes in a CO₂ incubator at about 36° C. to about 38° C. After incubation, the cells may be dislodged and the contents of the flask may be diluted with the same volume of TrypLE enzyme first used to coat the cells. The solution of TrypLE enzyme containing suspended cellular contents may then be transferred to a 50 ml centrifuge tube. DPBS without calcium or magnesium may be used to wash the contents of the flasks in volumes of about 5 ml for a T-25 flask, about 10 ml for a T-75 flask, and about 25 ml for a T-175 flask. The flasks used in the practice of the invention may be non-tissue culture treated flasks. About 50 ml of DPBS may be added to the 50 ml centrifuge tube containing the TrypLE enzyme and suspended cellular contents.

The 50 ml centrifuge tube may be centrifuged at about 120 g for about 5 minutes at ambient temperature. A small aliquot of the pellet, such as 20 ul may be removed to perform a cell count manually with a hemocytometer or automated apparatus. After centrifugation, the supernatant is removed and discarded, and the remaining pellet is suspended in about 7 ml of Chang's complete media.

The co-cultured and expanded cells suspended in the Chang' complete media may be prepared for cryopreservation, subjected to immunoselection for CD34 cells, prepared for infusion into a human, or prepared for cellular differentiation along any number of cell pathways.

Co-culture information may be obtained during the course of cell culture. Media may be changed every about 3 days or more. If co-culture contains more than 10,000,000 cells, then cells in the co-culture may be removed for subculturing under the same culture conditions or alternatively cryopreserved according to the cryopreservation methods described in this application.

Expansion of Cells through Culture on Plates

Cells may be co-cultured under sterile conditions using plating techniques. The media used in the methods of the invention may be Methocult 4034 which contains hSCF, hGM-CSF, hIL-3, hG-CSF, hEPO for the detection of BFU-E, CFU-GM, CFU-GEMM in CB. Culture media (MethoCult #4034) stored at −80° C. is thawed at about 2-8° C. or at room temperature. The thawed culture media and cells for co-culture are placed on ice for about 15 minutes prior to plating. About 0.3 ml of cellular suspension is added to the tube containing culture media, vortexed, and allowed to incubate on ice for about 30 minutes. Using a syringe, evenly distribute culture media in three wells of a four well culture plate at about 1 ml per well. Add about 1 ml of DPBS to the fourth well of the plate. The plate should be incubated under sterile conditions at about 37° C. for about 14 to about 21 days.

Flow Cytometry Analysis

Any sample of co-cultured cord blood cells and menstrual cells, whether or not expanded, may be analyzed for total cell count, cell viability by Trypan blue via dye exclusion, and expression of cell surface markers.

The total cell count and cell viability of expanded co-cultured cord blood cells and menstrual cells may be quantified by a hand count with a hemocytometer, a flow cytometer, or other means suitable for obtaining cell count, such as, for example, ViCell (Beckman Coulter) or software suitable to count cells displayed on a microscopic image.

Expanded co-cultured cord blood cells and menstrual cells may be analyzed by flow cytometry. 1×NH4CL lysing solution may be prepared from a StemKit by adding about 36 ml distilled water and 4 ml 10× lysing solution into a 50 ml tube. About 50 ul of cell sample may be added to two tubes to run the analysis. One tube is for CD34+/viability analysis and the second tube is for the isoclonic control. About 10 μL of 7-AAD Viability dye may be added to each tube. About 10 μL of CD45-FITC/CD34-PE may be added to the first tube. About 10 μL of CD45-FITC/CTRL-PE may be added to the second tube. The mixture may be vortexed and then incubated at about 15° C. to about 30° C. for at least 20 minutes protected from light. About 1 ml of 1×NH4CL lysing solution may then be added to each tube and vortexed. The mixture may be incubated at about 15° C. to about 30° C. for about 20 minutes. About 100 μL of Stem-Count Fluorospheres may be added to each tube and vortexed. The sample should then be run on a Flow Cytometer for analysis.

Expanded co-cultured cord blood cells and menstrual cells may also be analyzed by flow cytometry to analyze cell surface markers, cell viability, and other cell characteristics. Fresh samples of cells may also be analyzed according to the following protocol after cell lysis.

The sample of expanded co-cultured cord blood cells and menstrual cells may be centrifuged at about 2000 rpm for about seven minutes. The supernatant may be removed and the cells resuspended in about 100 ul of wash media (25% HSA, DNAse, Heparin, and HBSS w/Ca+ and Mg+). The resuspended cells may then be centrifuged in a Blood Bank Serofuge for about 1 minute. The supernatant may be decanted and the cells resuspended in about 1.2 ml Sheath fluid and vortexed.

The cells in Sheath fluid may be analyzed for any number of cell surface markers. As an example, and not a limitation, about a 100 ul sample of cells in Sheath fluid may be added to each tube containing the following reagents in either 10 ul or 20 ul volumes per reagent in each tube and then vortex the tube to mix the reagents and sample as described in Table A.

TABLE A
Flow Cytometry Load Schematic

| TUBE | FITC | PE | ECD | PC5 |
|---|---|---|---|---|
| 1 | IgG | IgG | IgG | IgG |
| 2 | HLA-I | CD133 | HLA-II | 7AAD |
| 3 | CD9 | CD54 | CD45 | CD10 |
| 4 | CD59 | CD63 | CD34 | CD13 |
| 5 | CD49e | CD81 | None | CD49f |
| 6 | CD44 | CD117 | None | CD38 |
| 7 | CD29 | CD105 | CD41 | CD3 |
| 8 | CD19 | CD166 | None | CD90 |
| 9 | NANOG | SSEA3 | None | 7AAD |
| 10 | CD14 | SSEA4 | None | 7AAD |
| 11 | None | CD56 | None | 7AAD |

Incubate at room temperature (15-30° C.) for 20 minutes. Protect from light. If running a fresh sample containing RBC's, add 500 μl of lyse solution and incubate at room temperature for another ten minutes and protect from light. If running a density gradient or a thawed sample, do not lyse. If sample was not lysed, wash after 20 minutes incubation with 1 ml of wash media. Centrifuge for 1 minute and then decant the supernatant. If sample was lysed, centrifuge sample for 1 minute and decant lyse. Add 1 ml of wash media, vortex, centrifuge again, and then decant again. Add 500 uL of Sheath fluid to each tube, vortex, and run on a FC500 Flow Cytometer.

Prior to cell marker analysis, a total cell count may be performed on cell samples. Any number of positive controls may be set up for flow cytometry analysis using a Kasumi-3 control cell or other control cells.

The materials for cell count and cell viability analysis include, but are not limited to, a flow cytometer, Isoflow Sheath Fluid, Coulter Clenz Cleaning Agent, and reagents including, but not limited to, CD45-FITC/CD34-PE, CD45-FITC/Isoclonic Control-PE, 7-AAD Viability Dye, Stem-Count Fluorospheres, Ammonium Chloride (NH4CL) Lysing Solution 10× Concentrated, and 22% Bovine Albumin Solution. See Stem Kit™ CD34+ HPC Enumeration Kit Package Insert—Version 03 (PNIM2390); Beckman Coulter Product Corrective Action, CXP 2.0 & 2.1 Panel Interruption-Mar. 10, 2006, PCA-M-D-1013; 14.3 StemLab, Build Number 200706260856, Version 3.2.1. Materials for Flow Cytometry also include, but are not limited to, Isoflow Sheath Fluid; Coulter Clenz Cleaning Agent; and the following reagents about 20-25° C. prior to use: CD117-PE, CD29-FITC, CD34-ECD, CD44-FITC, CD45-ECD, CD90-PC5, CD105-PE, CD166-PE, IgG-FITC, IgG-PE, IgG-ECD, IgGl-PC5, HLA-1-FITC, CD133-PE, HLA-II ECD, CD9-FITC, CD54-PE, CD10-PC5, CD59-FITC, CD63-PE, CD13-PC5, CD49e-FITC, CD81-PE, CD49f-PC5, CD44-FITC, CD38-PC5, CD29-FITC, CD105-PE, CD41-ECD, CD3-PC5, CD19-FITC, NANOG-FITC, SSEA3-PE, SSEA4-PE, CD14-FITC, CD56-PE, 7-AAD Viability Dye, Ammonium Chloride (NH4CL) Lysing Solution 10× Concentrated, Wash Media (comprising HBSS (Hanks with Ca+ and Mg+) 500 ml, Heparin 5 ml, Human Serum Albumin 25% 50 ml, DNASE-1 ampoule), Kasumi-3 cell line—CD34+ cells, Timer, and Vortex Mixer.

Cryopreservation of Expanded Cells

Co-cultured expanded cells suspended in the Chang's complete media obtained from tissue culture in flasks and also plates may be prepared for cryopreservation. The expanded cells may be resuspended in Chang's complete media. In an embodiment, the expanded cells may be combined with a cryopreservation agent in a 1:1 ratio. For example, a 5 ml vial may comprise about 2.5 ml of cells and 2.5 ml of cryopreservation agent. The suspension of expanded cells should be placed on ice for at least about 15 minutes before adding the cryopreservation agent.

The cryopreservation agent is prepared by combining ES-FBS and DMSO (99%) in a ratio of 4:1. For example, about 2 ml of ES-FBS may be added to 0.5 ml of DMSO. The ES-FBS may be chilled on ice for at least about 15 minutes before adding DMSO. Once chilled, the ES-FBS has DMSO added to it. The ES-FBS and DMSO may be chilled for at least about 15 minutes.

In an alternative embodiment, other cryopreservation media may be used. For example, cryopreservation agents may be used to maintain a high cell viability outcome post-thaw, such as, for example, CryoStor CS10 or CS5 (Biolife), embryonic cryopreservation media supplemented with propanediol and sucrose (Vitrolife), or SAGE media (Cooper Surgical). Glycerol may be used with other cryopreservation agents, such as, DMSO, or may be used alone at a concentration of about 10% in a media with suitable protein.

The cryopreservation agent may be added drop by drop to the suspension of expanded cells while on ice. The solution of expanded cells and suspended expanded cells may be mixed gently. The solution may aliquoted into desired volumes of vials in preparation for cryopreservation. The vials may be cryovials. The vials are maintained on ice until they are ready to be placed into a controlled rate freezer.

The preparation of expanded cells in cryopreservation agent may be exposed to several temperature reduction steps to reduce the temperature of the expanded cells to a final temperature of about −90° C. utilizing a controlled rate freezer or other suitable freezer system (dump-freeze monitored or a freeze container (Nalgene). Examples of control rate freezers include, but are not limited to, Cryomed Thermo Form a Controlled Rate Freezer 7454 (Thermo Electron, Corp.), Planar Controlled Rate Freezer Kryo 10/16 (TS Scientific), Gordinier, Bio-Cool-FTS Systems, and Asymptote EF600, BIOSTOR CBS 2100 series.

Temperature reduction steps may be programmed in the controlled rate freezer. The cryopreservation agent and expanded cells may be subjected to controlled rate temperature reductions in preparation for final storage in a freezer. The controlled rate reductions may be designed to maintain cell viability. A Cryo-Med Freezer (Thermo Electron Corp.), liquid nitrogen cylinder, and portable Cryo-Med Freezer may be used for controlled rate reductions in preparation for final storage in a freezer. The cells may be subject to controlled rate reductions in cryovials or cryobags to reach a temperature of about −90° C.

For a sample of expanded cells collected in a cryobag, the expanded cells may be subject to the following controlled rate reduction profile: wait at about 4° C., 1.0° C. per minute to −6.0° C. (sample), 25.0° C. per minute to −50.0° C. (chamber), 10.0° C. per minute to −14.0° C. (chamber), 1.0° C. per minute to −45.0° C. (chamber), 10.0° C. per minute to −90.0° C. (chamber), and end (sample at or below −85.0° C.).

For a sample of expanded cells collected in a cryovial, the cells may be subject to the following controlled rate reduction profile: wait at 4.0° C., 1.0° C. per minute to −3.0° C. (chamber), 10.0° C. per minute to −20.0° C. (chamber), 1.0° C. per minute to −40.0° C. (chamber), 10.0° C. per minute to −90.0° C. (chamber), and end.

Once the mixture of cryopreservation agent and expanded cells is at or below about −85° C., the cryopreservation vials are transferred to a cryogenic storage unit and stored in the vapor of liquid Nitrogen at a temperature at or below about −135° C. or alternatively vials may be stored in the liquid phase of liquid nitrogen. For example, a suitable cryogenic storage unit includes, but is not limited to, LN2 Freezer MVE 1830 (Chart Industries).

Immunoselection of Expanded Cells

Cells expanded through co-culture of cord blood cells and menstrual cells may be selected for at least one desired cell marker. By way of example, the desired cell marker may be CD34, HLA-II, or SSEA-4. Cells may also be subjected to a negative selection step to remove undesired cells. Throughout the cell selection process, the aseptic technique may be used. Cell selection may be used for fresh or thawed cells that were previously cryopreserved and co-cultured cells. Cell selections may be performed on as little as 2.5 million cells and up to 10 million cells. Cells may also be selected from a sample comprising less than 2.5 million cells or a sample comprising more than 10 million cells.

Materials for the cell selection include, but are not limited to, DNase, Pulmozyme (Genentech. Inc.)—1 ampoule, any Anti-cell surface mark to be selected negatively or positively for example, Anti-CD34 antibody, Goat anti-mouse IgG microbead, and magnetic field.

A cellular suspension comprising >=1.0×10⁶ cells of expanded co-cultured cord blood cells and menstrual cells at about 300 g for about 7 minutes at about 4° C. The supernatant may be removed without disturbing the cell pellet. The pellet may be resuspended with about 100 ul of wash media. In an embodiment, the anti-cell surface antibody is Anti-CD34 antibody. The cells in solution may be incubated on ice for about 20 minutes to about 25 minutes. After incubation, about 2 ml of wash media may be added to the cells and gently mixed. The mixture may be centrifuged for about 10 minutes at about 300 g at about 4° C. After centrifugation, the supernatant may be aspirated without disturbing the pellet. The pellet may be resuspended in about 80 ul of wash media. About 20 ul of goat anti-mouse IgG may be added to the cell suspension and gently mixed. The mixture may be incubated for about 30 minutes on ice. After incubation, the cells may be washed by adding about 2 ml of wash media and then mixing the solution. The cells may be centrifuged at about 300 g for about 10 minutes at about 4° C.

A column may be used to separate selected cells from the unselected cells. A column may be prepared by wetting it in about 500 ul of working buffer. After centrifugation, the supernatant may be aspirated without disturbing the pellet. The pellet may be resuspended in about 500 ul of working buffer. To avoid cell adherence, additional DNase may be added to the cells. The cellular suspension may be added to the column using a pipette. Antibody-labeled cells (positive fraction) should attach to the column subjected to a magnetic field provided by a MACS separator. Unlabeled cells (negative fraction) should flow through the column and be collected.

After the cellular suspension flows through the column and is collected as a negative fraction, the column may be washed at least 3 times using 500 ul of working buffer per wash. Each wash may completely flow through the column prior to the next wash. Each wash may be collected with the negative fraction. About 100 ul of the negative fraction may be removed for analysis. A cell count using the Hemacytometer and viability using Trypan Blue or another method may be performed. Phenotype analysis may occur using flow cytometry as previously discussed or using another flow cytometry method. The negative fraction may be prepared for cryopreservation or placed in culture for further cell growth and expansion and later processing.

After the negative fraction is collected and the column is washed, another tube may be placed under the column to collect the positive fraction. About one ml of working buffer may be added to the column and remove the magnetic field form the column. The working buffer and positive fraction should be collected. A plunger may be used to remove as many labeled cells as possible from the column for the positive fraction. About 100 ul of the positive fraction may be removed for analysis including, but not limited to, cell count and viability using Trypan Blue or flow cytometry. The positive fraction may be cryopreserved, cultured, or prepared for therapeutic use.

The step of immunoselecting cells expressing desired cell markers may occur according to the embodiment of the invention as shown in FIG. 1. In particular, the selection may occur after at least the step of co-culturing the cord blood cells and the menstrual cells. The steps of selecting menstrual stem cells expressing certain cell markers provides a population of enriched cells expressing the selected cell marker, which may be used for further cell culture, cryopreservation, or therapeutic use.

In an embodiment, the step of selecting expanded cells expressing CD34 from a population of cells comprises labeling expanded cells with anti-human CD34 antibodies and then labeling the CD34 stem cell-anti-human CD34 antibody complexes with magnetically-labeled antibodies capable of binding to the anti-human CD34 antibodies. Additionally, the method comprises labeling any cell expressing CD34 with anti-human CD34 antibodies and then labeling the CD34 cell-anti-human CD34 antibody complexes with magnetically-labeled antibodies capable of binding to the anti-human CD34 antibodies. The method of selecting cells expressing CD34 may include selecting any cell expressing CD34 implemented or expanded according to the invention. The step of immunoselecting cells comprises exposing the complexes comprising CD34 cells, anti-human CD34 antibodies, and magnetically-labeled antibodies to a magnetic field to draw the magnetically-labeled antibodies and the rest of the complex to the column and washing all other CD34 negative cells through the column for analysis.

Throughout the steps of selecting cells expressing CD34, the cellular suspension of cells and working buffer (MACS® Separation running buffer with DNase, Miltenyi) may be maintained at a cold temperature. Other magnetic separation kits may be suitable for use (R&D Systems).

The cellular suspension may be centrifuged at about 300 g for about 10 minutes. The pellet may be suspended in a working buffer with anti-human CD34 antibodies. For example, the working buffer may comprise, for example, PBS at about pH 7.2, bovine serum albumin, EDTA and about 0.09% Azide (or suitable solution) (BD Biosciences). The pellet may be suspended, for example, in about 100 ul of working buffer and about 5 ug of purified antibodies having affinity for human CD34. The antibody may be monoclonal or polyclonal. The antibody may be purified IgG or other antibody capable of binding human CD34. The antibody may be a mouse anti-CD34 antibody.

The Solution comprising the cells, working buffer and anti-CD34 antibodies are incubated for an incubation period. For example, the incubation period may comprise between about 20 minutes to about 25 minutes on ice. The incubation period may, alternatively, be shortened to less than about 20 minutes if the temperature is at least about 2° C. to about 8° C. or about 5 minutes to about 10 minutes if at least at room temperature. After the incubation period, the solution with the cells may be washed with working buffer to remove unbound antibody and then centrifuged. For example, the centrifugation may occur at about 300 g for about 10 minutes. After centrifugation, the supernatant is aspirated and may be saved for analysis, and the pellet is suspended in working buffer. For example, the volume of the working buffer may be about 80 ul.

A second batch of antibodies having microbeads affixed thereto and having an affinity for the anti-human CD34 antibody are added to the working buffer used to suspend the pellet. The microbeads may comprise, for example, iron oxide and polysaccharide. The microbeads may be biodegradable. The microbeads are available through Miltenyi Biotec. For example, the second batch of antibodies are specific for an antibody having affinity for human CD34, such as, for example, goat anti-mouse IgG antibody. The antibody may be monoclonal or polyclonal. The antibody may be capable of binding to the light chain and/or the heavy chain of mouse antibodies. The antibody may be for example a goat anti-mouse IgG microbead conjugate available through Miltenyi Biotec as product 130-048-401. A two (2) ml vial of the aforementioned goat anti-mouse IgG may be used for approximately 1.0×10̂9 of total unseparated cells.

The cellular suspension is incubated for a second incubation period. For example, the incubation period may be in a range of about 30 minutes to about 35 minutes. Alternatively, the incubation period may be less than about 30 minutes where the incubation occurs at about 2° C. to about 8° C. or about 5 to about 10 minutes where incubation occurs at about room temperature. After the incubation period is complete, the cells are washed with working buffer, such as for example, about 2 ml of working buffer, and the cells are then centrifuged. For example, the centrifugation may occur at about 300 g for about 10 minutes. The supernatant may be aspirated and saved for analysis, and the pellet containing cells is suspended in working buffer, such as for example, about 500 ul of working buffer.

Cell Separation

The CD34 cells may be separated from a cellular suspension in working buffer using an MS column to separate the CD34 stem cells. For example, an MS Column (Miltenyi Biotec) or other suitable column may be used. Alternatively, other suitable methods to separate cells may be used. A MiniMACS kit available through Miltenyi Biotec comprising a unit, multistand, MS columns and microbeads may be used for CD34 cell selection. The MS column may be prepared by rinsing it with working buffer. For example, the volume of working buffer used to rinse the column may be about 500 ul. The column is placed in a magnetic field of a MACS separator available through Miltenyi Biotec or suitable separator providing a magnetic field.

The cellular suspension in working buffer is added to the column with a pipette or other device capable of transferring a volume of liquid. The CD34 cells labeled with anti-human CD34 antibodies, which are bound with antibodies attached to microbeads, are held in the column due to the magnetic field of the MACS separator. Any unlabeled cells, along with the working buffer, should flow through the column and may be collected in a sterile tube for cell phenotyping and cell count. The unlabeled cells, which flow through the column, may be identified as a negative fraction. The column may be washed with working buffer after adding the cellular suspension. For example, the column may be washed at least 3 times or any amount of time that causes all or substantially all of the unlabeled cells to pass through the column. The effluent from the washing steps may be collected for cell phenotyping and count. The effluent may also be identified as a negative fraction.

The labeled CD34 cells may be collected from the column after the column is washed. The labeled CD34 cells are collected by placing a sterile tube under the column and removing the column from the magnetic field. Once the column is removed from the magnetic field, the labeled CD34 cells pass through the column and into the sterile tube. Residual labeled CD34 cells in the column may be washed out by adding working buffer to the column to wash the cells through the column and, optionally, by stripping the column with a plunger to release the cells. The collected labeled CD34 cells may be identified as the positive fraction. In order to obtain a more purified population of labeled CD34 cells, the positive fraction may, optionally, be run through a column at least one more time following the previously disclosed washing procedure. The positive fraction may be centrifuged at about 300 g for about 10 minutes and the supernatant aspirated. The pellet may be suspended in about 5 ml of working buffer.

The positive fraction and the negative fraction are analyzed with a hemocytometer to obtain a total count of viable cells. The negative fraction is analyzed by flow cytometry for phenotyping. Optionally, the positive fraction may be phenotyped using flow cytometry.

The positive fraction containing cells expressing CD34 or other desired cell marker, may be prepared for cryopreservation in accordance with the methods of the invention. About one ml of human serum albumin, about 3 ml of DPBS and about one ml of DMSO are added to the about 5 ml of the positive fraction. Alternatively, other culture media may be used in the step of preparing cells for cryopreservation, such as, for example, complete media, bovine serum albumin, fetal calf serum, fetal bovine serum, protein plasma fraction, or autologous serum. The solution containing expanded cells is mixed and cooled on ice for about 10 minutes. About one ml of DMSO is added as a cryopreservative. Alternatively, about 1 ml of a mixture of about 6% HES hydroxyethyl starch and about 5% DMSO may be used as a cryopreservative. The resulting solution is aliquoted into cryovials. Alternatively, the resulting solution may be aliquoted into any container suitable for cryopreservation, such as, for example, a cryopreservation bag. The cryovials are then cryopreserved in a controlled rate freezer (Cryomed) in accordance with controlled rate freezer protocol of the invention. Once the solution containing expanded cells reaches the target temperature of about −90° C., the cryovials are transferred into a long term storage freezer and stored at about −135° C. or less. Alternatively, the cryovials or other suitable cryopreservation container may be placed into a monitored dump freeze and frozen to about −80° C. and then transferred into the vapor phase of liquid nitrogen in a long term storage freezer at about −135° C. or less.

Therapeutic Use of Expanded Cells

Expanded cells expressing CD34 obtained by the invention may be prepared for therapeutic use to treat human disorders. In an embodiment, expanded cells, immunoselected CD34 cells from expansion through co-culture, or expanded cells that have been thawed after cryopreservation may be prepared for intravenous infusion into a recipient.

The techniques for intravenous infusion may occur according to practices acceptable for cellular infusion into humans. The intravenous infusion may involve autologous or allogenic infusion of expanded CD34 cells.

Differentiation of Expanded Cells

The expanded CD34 cord blood cells of the invention may be capable of differentiating into any of the 260 somatic cells in the body. For example, the cells may be able to differentiate into at least hepatic, pancreatic, myogenic, osteogenic, chondrogenic, adipocytic, epithelial, neural, keratinocytes, and cardiomyocytes. The cells may also possess the capacity to differentiate when cultured with other predisposed cells, such as hepatic, pancreatic, myogenic, osteogenic, chondrogenic, adipocytic, epithelial, neural, keratinocytes, and cardiomyocytes, as seen in a co-culture system. Cells obtained from differentiation and cells obtained from co-culture may also have the potential to be used in treatment for replacement or regeneration therapies, other therapeutic applications, cosmeceuticals, organ rejection therapies, and other applications.

Expanded cord blood cells expressing CD34 obtained by the invention may be prepared for differentiation into a specific cell lineage. In an embodiment, expanded cells, immunoselected CD34 cells from expansion through co-culture, or expanded cells that have been thawed after cryopreservation may be prepared for cellular differentiation.

The techniques for differentiation may occur according to practices acceptable for cellular differentiation in humans.

The following examples are offered by way of illustration and not by way of limitation. Those skilled in the art will recognize that variations of the invention embodied in the examples can be made, especially in light of the teachings of the various references cited herein, the disclosures of which are incorporated by reference in their entirety.

### EXAMPLE 1

### Culture of Cord 871R

Cord blood cells 871R were collected according to the methods described in the application and used in the cord blood collection industry.

The cord blood sample for Cord 871R cells was processed about 2 days after collection and cryopreserved according to the processing and cryopreservation methods for cord blood described in this application. Cord 871R cells were held in cryopreservation for about two and a half years. Cord 871R cells were thawed according to the thawing methods described in this application.

Culture—Plate

About 3 ml of culture media (Methocult #4034—semi-solid media) was thawed at room temperature and then placed on ice for 15 minutes along with cord cell dilution (500,000 cells/mL). After 15 minutes about 0.3 ml of the cord cell dilution was inoculated into the tube of the culture media. The tube was gently vortexed and then incubated on ice for about 30 minutes. After incubation the culture media and Cord 871R cells were evenly divided into the first 3 wells of a four well culture plate. One milliliter of DPBS was added to the fourth well to assist with humidity and the cells were incubated at 37° C. Certain results of cell culture are summarized in TABLE B.

Culture—Flask

Cord 871R cells were subjected to the step of thawing cryopreserved cells and then washing them in Chang's complete media through centrifugation disclosed in this application. The pellet of cells was resuspended in about 7 ml of 15% Chang's Complete media.

The resuspended Cord 871R cells were seeded at about 1,000,000 cells in 7 ml of 15% Chang's Complete media in a T25 non-tissue treated culture flask. The cells were incubated in a CO₂ incubator at about 36° C. to about 38° C. There were no adherent cells at the first passage.

TABLE B
Plate Culture of M28100RM, M28100RM, M28101R, M28101R +
871R, and 871R

| 10,000 | | | | | |
|---|---|---|---|---|---|
| per | | M28100RM + | | M28101R + | |
| well | M28100RM | 871R | M28101R | 871R | 871R |
| BFU-E | 2 | 0.67 | 0 | 0 | 0 |
| AVER- | | | | | |
| AGE | | | | | |
| CFU- | 0.67 | 3.67 | 0.33 | 0 | 0 |
| GM | | | | | |
| AVER- | | | | | |
| AGE | | | | | |
| CFU- | 0 | 0 | 0 | 0 | 0 |
| GEMM | | | | | |
| AVER- | | | | | |
| AGE | | | | | |

### EXAMPLE 2

### Culture of M28100RM Menstrual Cells

Menstrual stem cells M28100RM were collected according to the methods of U.S. Patent Publication No. 20080241113 by collecting about 9 ml of menstrual flow and shipping it to a processing facility within about 24 hours to about 48 hours of collection in procurement media DPBS without antibiotics with calcium and magnesium, and preservation-free Heparin. The menstrual flow sample was incubated in antibiotics for 24 hours, and subsequently washed, concentrated through centrifugation, mixed with 10% DMSO cryopreservation agent, and cryopreserved according to the teachings of U.S. Patent Application Publication No. 20080241113.

The menstrual flow sample for M28100RM menstrual cells was processed and the M28100RM menstraul cells were cryopreserved about 2 days after collection. The cells were held in cryopreservation for about 8 months and then thawed for CFU according to the methods described in this application.

Culture—Plate

Three 3 ml tubes of culture media (MethoCult #4034-semi-solid media) were thawed at room temperature and then placed on ice for 15 minutes at cell dilutions of 50,000 cells/ml, 100,000 cells/ml, and 21,600 cells/ml. After 15 minutes, about 0.3 ml of each menstrual cell dilution was inoculated into a tube of Methocult #4034 semi-solid media. The tubes were gently vortexed and then incubated on ice for 30 minutes. After incubation, the culture media and menstrual stem cells were evenly divided into the first 3 wells of a 4-well plate. One ml of DPBS was added to the fourth well to assist with humidity, and the cells were incubated at 37° C. Certain results of cell culture are summarized in TABLE B.

Culture—Flask

M28100RM menstrual cells were subjected to the step of thawing cryopreserved cells and then washing them in Chang's complete media through centrifugation disclosed in this application. The pellet of cells was resuspended in about 25 ml of 15% Chang's complete media.

The resuspended M28100RM menstrual cells were seeded at about 221,000 cells in 7 ml of 15% Chang's Complete media in a T25 non-tissue culture treated flask. The cells were incubated in a CO₂ incubator at about 36° C. to about 38° C. until the cells were confluent at about 70-80%. The cells went through several passages shown on TABLE C. Passages occurred after about three or four days comprising a complete change of the 15% Chang's Complete media shown in TABLE C.

TABLE C
Culture of M28100RM Menstrual Cells

| | | | | | | | PD |
|---|---|---|---|---|---|---|---|
| | Total | | | | | | Time |
| Passage | Count | Flow | Cryo | Culture | Days | Doubled | (hrs) |
| P1 | 722,656 | | | 722,656 | 0 | | |
| P2 | 917,500 | | | 917,500 | 4 | 1.27 | |
| P3 | 1,136,000 | | | 1,136,000 | 2 | 1.24 | 38.77 |
| P4 | 8,960,000 | | | 60,000 | 5 | 7.89 | 15.21 |
| P5 | 1,060,000 | | | 1,060,000 | 6 | 17.67 | 8.15 |

### EXAMPLE 3

### Culture of M28101R

Menstrual stem cells M28100RM were collected according to the methods of U.S. Patent Publication No. 20080241113 by collecting about 9 ml of menstrual flow and shipping it to a processing facility within about 24 hours to about 48 hours of collection in procurement media DPBS without antibiotics with calcium and magnesium, and preservation-free Heparin. The menstrual flow sample was incubated in antibiotics for 24 hours, and subsequently washed, concentrated through centrifugation, mixed with 10% DMSO cryopreservation agent, and cryopreserved according to the teachings of U.S. Patent Application Publication No. 20080241113.

The menstrual flow for M28101R menstrual cells was processed after collection and cryopreserved according to the menstrual stem cell processing and cryopreservation methods described in this application about 3 days after collection. The cells were held in cryopleservation for about seven months. M28101R menstrual cells were thawed according to the thawing methods described in this application.

Culture—Plate

Three 3 ml tubes of culture media (MethoCult #4034-semi-solid media) were thawed at room temperature and then placed on ice for 15 minutes along with the cell dilutions (50,000 cells/mil, 100,000 cells/ml, and 216,000 cells/mil) of the M28101R menstrual cells. After 15 minutes, 0.3 ml of each menstrual cell dilution was inoculated into a tube of culture media. The tubes were then gently vortexed and then incubated on ice for 30 minutes. After incubation, the menstrual cells in culture media were evenly divided into the first 3 wells of a 4-well plate. One milliliter of DPBS was added to the fourth well to assist with humidity and the cells were incubated at 37° C. Certain results of cell culture are summarized in TABLE B.

Culture—Flask

M28101R menstrual cells were subjected to the step of thawing cryopreserved cells and then washing them in Chang's complete media through centrifugation disclosed in this application. The pellet of cells was resuspended in about 25 ml of 15% Chang's complete media.

The resuspended menstrual cells were seeded at about 724,780 cells in 7 ml of 15% Chang's Complete media in a T25 non-tissue culture treated flask. The cells were incubated in a CO₂ incubator at about 36° C. to about 38° C. until the cells are confluent at about 70-80%. The cells went through several passages shown in TABLE D. Passages occurred after about three or four days comprising a complete change of the 15% Chang's Complete media shown in TABLE D.

TABLE D
Culture of M28101R Menstrual Cells

| | Total | | | | | | PD Time |
|---|---|---|---|---|---|---|---|
| Passage | Count | Flow | Cryo | Culture | Days | Doubled | (hrs) |
| P1 | 2,170,000 | | | 2,170,000 | 0 | | |
| P2 | 18,200,000 | | 17,800,000 | 456,000 | 4 | 4.76 | 20.17 |
| P3 | 3,760,000 | | 3,500,000 | 260,000 | 4 | 1.73 | 55.40 |
| P4 | 1,619,940 | | | 1,619,940 | 5 | 6.23 | 19.26 |
| P5 | 5,170,000 | | 5,032,000 | 136,000 | 3 | 3.19 | 22.56 |

### EXAMPLE 4

### Culture of M28100RM Menstrual Cells and Cord871R

Menstrual stem cells M28100RM were collected according to the methods of U.S. Patent Publication No. 20080241113 by collecting about 10 ml of menstrual flow and shipping it to a processing facility within about 24 hours to about 48 hours of collection in procurement media DPBS without antibiotics with calcium and magnesium, and preservation-free Heparin. The menstrual flow sample was incubated in antibiotics for 24 hours, and subsequently washed, concentrated through centrifugation, mixed with 10% DMSO cryopreservation agent, and cryopreserved according to the teachings of U.S. Patent Application Publication No. 20080241113.

The menstrual flow for menstrual stem cells M28100R was processed after collection and cryopreserved according to the menstrual stem cell processing and cryopreservation methods described in this application about 2 days after collection. The cells were held in cryopreservation for about six months. M28100R cells were thawed according to the thawing methods described in this application.

The cord blood sample for Cord871R cells was processed about 2 days after collection and cryopreserved according to the processing and cryopreservation methods for cord blood described in this application. The Cord871R cells were held in cryopreservation for about two and a half years. Cord871R cells were thawed according to the thawing methods described in this application.

Culture—Plate

Three 3 ml tubes of culture media (MethoCult #4034-semi-solid media) were thawed at room temperature and then placed on ice for 15 minutes along with the cell dilutions (50,000 M28100R cells/ml+500,000 Cord871R cells/ml; 100,000 M28100R cells/ml+500,000 Cord871R cells/ml; and 216,000 M28100R cells/ml+500,000 Cord871R cells/ml). After 15 minutes 0.3 mL of each cell dilution in culture media was inoculated into separate tubes of culture media. The tubes were then gently vortexed and then incubated on ice for 30 minutes. After incubation, each cell dilution in culture media was evenly divided into the first 3 wells of separate 4-well plates. One ml of DPBS was added to the fourth well of each plate to assist with humidity and the cells were incubated at 37° C. Certain results of cell culture are summarized in TABLE B.

Culture—Flask

Cord 871R cells and M28101R menstrual cells were separately subjected to the step of thawing cryopreserved cells and then washing them in Chang's complete media through centrifugation disclosed in this application. The pellet of cells was resuspended in about 7 ml of 15% Chang's complete media.

The resuspended M28101R menstrual cells were seeded at about 221,400 cells with about 1,000,000 Cord 871R cells in 7 ml of 15% Chang's Complete media in a T25 non-tissue culture treated flask. The cells were incubated in a CO₂ incubator at about 36° C. to about 38° C. until the cells were confluent at about 70-80%. The cells went through several passages shown on TABLE E. Passages occurred after about three or four days comprising a complete change of the 15% Chang's Complete media shown in TABLE E.

TABLE E
Culture of M28101R Menstrual Cells and Cord871R Cells

| | Total | | | | | | |
|---|---|---|---|---|---|---|---|
| Passage | Count | Flow | Cryo | Culture | Days | Doubled | PD Time (hrs) |
| P1 | 341,028 | | | 341,028 | 0 | | |
| P2 | 478,275 | | | 478,275 | 4 | 1.40 | 68.45 |
| P3 | 718,622 | | | 718,622 | 4 | 1.50 | 63.89 |
| P4 | 1,700,037 | | | 1,700,037 | 5 | 2.37 | 50.73 |
| P5 | 5,080,000 | 4,950,000 | | 127,000 | 6 | 84.67 | 1.70 |

### EXAMPLE 5

### Culture of M28101R+Cord 871R

Menstrual stem cells M28101R were collected according to the methods of U.S. Patent Publication No. 20080241113 by collecting about 9 ml of menstrual flow and shipping it to a processing facility within about 24 hours to about 48 hours of collection in procurement media DPBS without antibiotics with calcium and magnesium, and preservation-free Heparin. The menstrual flow sample was incubated in antibiotics for 24 hours, and subsequently washed, concentrated through centrifugation, mixed with 10% DMSO cryopreservation agent, and cryopreserved according to the teachings of U.S. Patent Application Publication No. 20080241113.

The menstrual flow for menstrual stem cells M28101R was processed after collection and cryopreserved according to the menstrual stem cell processing and cryopreservation methods described in this application about 3 days after collection. The cells were held in cryopreservation for about six months. M28101R cells were thawed according to the thawing methods described in this application.

The cord blood sample for Cord 871R cells was processed about 2 days after collection and cryopreserved according to the processing and cryopreservation methods for cord blood described in this application. The Cord 871R cells were held in cryopreservation for about two and a half years. Cord 871R cells were thawed according to the thawing methods described in this application.

Culture—Plate

Three 3 ml tubes of culture media (MethoCult #4034-semi-solid media) were thawed at room temperature and then placed on ice for 15 minutes along with the cell dilutions (50,000 M28101R cells/ml+500,000 Cord 871R cells/ml; 100,000 M28101R cells/ml+500,000 Cord 871R cells/ml; and 216,000 M28101R cells/ml+500,000 Cord871R cells/ml). After 15 minutes 0.3 ml of each cell dilution in culture media was inoculated into separate tubes of culture media. The tubes were then gently vortexed and then incubated on ice for 30 minutes. After incubation, each cell dilution in culture media was evenly divided into the first 3 wells of separate 4-well plates. One ml of DPBS was added to the fourth well of each plate to assist with humidity and the cells were incubated at 37° C. Certain results of cell culture are summarized on TABLE B.

Culture—Flask

Cord 871R cells and M28101R menstrual cells were subjected to the step of thawing cryopreserved cells and then washing them in Chang's complete media through centrifugation disclosed in this application. The pellet of cells was resuspended in about 7 ml of 15% Chang's complete media.

The resuspended menstrual cells were seeded at about 724,000 cells with about 1,000,000 cord blood stem cells in 7 ml of 15% Chang's Complete media in a T25 non-tissue culture treated flask. The cells were incubated in a CO₂ incubator at about 36° C. to about 38° C. until the cells are confluent at about 70-80%. The cells went through several passages shown on TABLE F. Passages occurred after about three or four days comprising a complete change of the 15% Chang's Complete media shown in TABLE F.

TABLE F
Culture of M28101R Menstrual Cells + Cord871R Cell

| | | | | Seeded in | | | |
|---|---|---|---|---|---|---|---|
| Passage | Total Count | Flow | Cryo | Culture | Days | Doubled | PD Time (hrs) |
| P1 | 1,200,000 | | | 1,200,000 | 0 | | |
| P2 | 18,430,000 | | 17,800,000 | 576,000 | 4 | 15.36 | 6.25 |
| P3 | 3,380,000 | | | 3,380,000 | 4 | 5.87 | 16.36 |
| P4 | 12,430,000 | | 12,000,000 | 430,000 | 3 | 36.78 | 1.96 |
| P5 | 1,450,000 | | | 1,450,000 | 3 | 3.37 | 21.35 |

### EXAMPLE 6

### Culture of M2-048

Menstrual stem cells M2-048 were collected according to the methods of U.S. Patent Publication No. 20080241113 by collecting about 9.7 ml of menstrual flow and shipping it to a processing facility within about 24 hours to about 48 hours of collection in procurement media DPBS without antibiotics with calcium and magnesium, and preservation-free Heparin. The menstrual flow sample was incubated in antibiotics for 24 hours, and subsequently washed, concentrated through centrifugation, mixed with 10% DMSO cryopreservation agent, and cryopreserved according to the teachings of U.S. Patent Application Publication No. 20080241113.

The menstrual flow for menstrual stem cells M2-048 was processed after collection and cryopreserved according to the menstrual stem cell processing and cryopreservation methods described in this application about 3 days after collection. The cells were held in cryopreservation for about three months. M2-048 cells were thawed according to the thawing methods described in this application.

Culture—Plate

Three 3 ml tubes of culture media (MethoCult #4034-semi-solid media) were thawed at room temperature and then placed on ice for 15 minutes at cell dilutions of 50,000 cells/ml, 100,000 cells/ml, and 216,000 cells/ml. After 15 minutes, about 0.3 ml of each M2 cell dilution was inoculated into a tube of Methocult #4034 semi-solid media. The tubes were gently vortexed and then incubated on ice for 30 minutes. After incubation, the culture media and menstrual stem cells were evenly divided into the first 3 wells of a 4-well plate. One ml of DPBS was added to the fourth well to assist with humidity, and the cells were incubated at 37° C.

Culture—Flask

M2-048 menstrual cells were subjected to the step of thawing cryopreserved cells and then washing them in Chang's complete media through centrifugation disclosed in this application. The pellet of cells was resuspended in about 7 ml of 15% Chang's complete media.

The resuspended menstrual cells were seeded at about 1,000,000 cells in 7 ml of 15% Chang's Complete media in a T25 non-tissue culture treated flask. The cells were incubated in a CO₂ incubator at about 36° C. to about 38° C. until the cells were confluent at about 70-80%. The cells went through several passages shown on TABLE G. Passages occurred after about three or four days comprising a complete change of the 15% Chang's Complete media shown in TABLE G.

TABLE G
Culture of M2-048 Menstrual Cells

| | | | | Seeded | | | |
|---|---|---|---|---|---|---|---|
| | | | | for Next | | | |
| Passage | Total Count | Flow | Cryo | Passage | Days | Doubled | PD Time (hrs) |
| P7 | 1,100,000 | | 1,030,000 | 64,500 | 3 | | |
| P8 | 866,700 | | | 866,700 | 4 | 13.44 | 7.14 |
| P9 | 6,640,000 | 4,180,000 | 2,340,000 | 123,000 | 3 | 7.66 | 9.40 |
| P10 | 855,000 | | | 855,000 | 3 | 6.95 | 10.36 |
| P11 | 12,620,000 | 4,780,000 | 7,610,000 | 217,500 | 4 | 14.76 | 6.50 |
| P12 | 1,620,000 | | 1,530,000 | 89,976 | 3 | 7.45 | 9.67 |
| P13 | 110,200 | | | 110,200 | 4 | 1.22 | 78.38 |
| P14 | 9,940,000 | 4,090,000 | 5,840,000 | 0 | 4 | 90.20 | 1.06 |

### EXAMPLE 7

### Culture of M2-048+Mixed Cord (5006-2180 and 5013-2670)

Menstrual stem cells M2-048 were collected according to the methods of U.S. Patent Publication No. 20080241113 by collecting menstrual flow and shipping it to a processing facility within 48 hours of collection in procurement media DPBS without antibiotics with calcium and magnesium, and preservation-free Heparin. The menstrual flow sample was incubated in antibiotics for 24 hours, and subsequently washed, concentrated through centrifugation, mixed with 10% DMSO cryopreservation agent, and cryopreserved according to according to the teachings of U.S. Patent Application Publication No. 20080241113.

The menstrual flow for menstrual stem cells M2-048 was processed after collection and cryopreserved according to the menstrual stem cell processing and cryopreservation methods described in this application about 3 days after collection. The cells were held in cryopreservation for about three months. M2-048 cells were thawed according to the thawing methods described in this application.

The cord blood sample for Cord 5006-2180 cells was processed about 1 day after collection and cryopreserved according to the processing and cryopreservation methods for cord blood described in this application. The Cord 5006-2180 cells were held in cryopreservation for about three years. Cord 5006-2180 cells were thawed according to the thawing methods described in this application.

The cord blood sample for Cord 5013-2670 cells was processed about 1 day after collection and cryopreserved according to the processing and cryopreservation methods for cord blood described in this application. The Cord 5013-2670 cells were held in cryopreservation for about three years. Cord 5013-2670 cells were thawed according to the thawing methods described in this application.

Culture—Plate

One 3 ml tube of culture media (MethoCult #4034-semi-solid media) were thawed at room temperature and then placed on ice for 15 minutes with Cord 5006-2180 cell dilutions of 50,000 cells/ml, 100,000 cells/ml, and 216,000 cells/ml. After 15 minutes, about 0.3 ml of each cell dilution was inoculated into a tube of Methocult #4034 semi-solid media. The tubes were gently vortexed and then incubated on ice for 30 minutes. After incubation, the culture media and menstrual stem cells were evenly divided into the first 3 wells of a 4-well plate. One ml of DPBS was added to the fourth well to assist with humidity, and the cells were incubated at 37° C.

Culture—Flask

The resuspended Cord 5006-2180 cells were seeded at about 2,000,000 cells in 7 ml of 15% Chang's Complete media in two T25 non-tissue culture treated flasks. The cells were incubated in a CO₂ incubator at about 36° C. to about 38° C. until the cells are confluent at about 70-80%. The cells went through several passages. Passages occurred after about three or four days comprising a complete change of the 15% Chang's Complete media.

Culture—Plate

Two 3 ml tubes of culture media (MethoCult #4034-semi-solid media) were thawed at room temperature and then placed on ice for 15 minutes and Cord 5013-2670 cell dilutions of 50,000 cells/mL, 100,000 cells/mL, and 216,000 cells/mL. After 15 minutes, about 0.3 ml of each M2 cell dilution was inoculated into a tube of Methocult #4034 semi-solid media. The tubes were gently vortexed and then incubated on ice for 30 minutes. After incubation, the culture media and menstrual stem cells were evenly divided into the first 3 wells of a 4-well plate. One ml of DPBS was added to the fourth well to assist with humidity, and the cells were incubated at 37° C.

TABLE I
Plate Culture of Cord 5013-2670

| | Quadrant | Quadrant | Quadrant | Quadrant | |
|---|---|---|---|---|---|
| | 1 | 2 | 3 | 4 | Total |
| Well 1 BFU-E | 2 | 0 | 0 | 0 | 2 |
| Well 2 BFU-E | 0 | 0 | 2 | 0 | 2 |
| Well 3 BFU-E | 0 | 1 | 0 | 0 | 1 |
| Avg_{TOTAL} BFU-E | | | | | 1.6 |
| Well 1 GM | 0 | 0 | 1 | 0 | 1 |
| Well 2 GM | 0 | 0 | 0 | 0 | 0 |
| Well 3 GM | 0 | 0 | 0 | 0 | 0 |
| Avg_{TOTAL} GM | | | | | 0.3 |
| Well 1 GEMM | 5 | 0 | 1 | 5 | 11 |
| Well 2 GEMM | 0 | 3 | 3 | 7 | 13 |
| Well 3 GEMM | 5 | 3 | 7 | 0 | 15 |
| Avg_{TOTAL} | | | | | 13 |
| GEMM | | | | | |

Culture—Flask

Cord 5013-2670 cells were subjected to the step of thawing cryopreserved cells and then washing them in Chang's complete media through centrifugation disclosed in this application. The pellet of cells was resuspended in about 7 ml of 15% Chang's complete media.

The resuspended menstrual cells were seeded at about 2,000,000 cells in 7 ml of 15% Chang's Complete media in two T25 non-tissue culture treated flasks. The cells were incubated in a CO₂ incubator at about 36° C. to about 38° C. until the cells are confluent at about 70-80%. The cells went through several passages. Passages occurred after about three or four days comprising a complete change of the 15% Chang's Complete media.

Culture—Plate

Three 3 ml tubes of culture media (MethoCult #4034-semi-solid media) were thawed at room temperature and then placed on ice for 15 minutes along with the cell dilutions (10,000 M2-048 menstrual cells+250,000 Cord 5006-2180 cells/ml; 10,000 M2-048 menstrual cells+250,000 Cord 5013-2670 cells/ml; and 10,000 M2-048 menstrual cells+500,000 Cord 5013-2670 cells/ml). After 15 minutes, about 0.3 ml of each cell dilution was inoculated into separate tubes of Methocult #4034 semi-solid media. The tubes were gently vortexed and then incubated on ice for 30 minutes. After incubation, the culture media and menstrual stem cells were evenly divided into the first 3 wells of a 4-well plate. One ml of DPBS was added to the fourth well to assist with humidity, and the cells were incubated at 37° C.

TABLE J
Plate Culture of Cord 5013-2670 + M2-048

| | Quadrant | Quadrant | Quadrant | Quadrant | |
|---|---|---|---|---|---|
| | 1 | 2 | 3 | 4 | Total |
| Well 1 BFU-E | 1 | 5 | 1 | 0 | 7 |
| Well 2 BFU-E | 0 | 0 | 0 | 12 | 12 |
| Well 3 BFU-E | 0 | 0 | 0 | 0 | 0 |
| Avg_{TOTAL} BFU-E | | | | | 6.3 |
| Well 1 GM | 0 | 2 | 2 | 1 | 5 |
| Well 2 GM | 0 | 1 | 3 | 0 | 4 |
| Well 3 GM | 0 | 0 | 0 | 0 | 0 |
| Avg_{TOTAL} GM | | | | | 3 |
| Well 1 GEMM | 0 | 0 | 0 | 2 | 2 |
| Well 2 GEMM | 0 | 2 | 0 | 0 | 2 |
| Well 3 GEMM | 0 | 0 | 0 | 1 | 1 |
| Avg_{TOTAL} | | | | | 1.6 |
| GEMM | | | | | |

Culture—Flask

1,000,000 M2-048 menstrual cells taken from passage 4 and 100,000 Cord 5006-2180 cells were seeded in 7 ml of 15% Chang's Complete media in a T25 non-tissue culture treated flask. 1,000,000 M2-048 menstrual cells and 1,000,000 Cord 5013-2670 cells were seeded in 7 ml of 15% Chang's Complete media. After trypsinizations, the two cell cultures were mixed as M2-048 and mixed Cord 5006-2180 with Cord 5013-2670. The cells were incubated in a CO₂ incubator at about 36° C. to about 38° C. until the cells are confluent at about 70-80%. The cells went through several passages. Passages occurred after about three or four days comprising a complete change of the 15% Chang's Complete media.

TABLE K
Culture: M2-048-01 P4 + mixed Cord 5006-2180 & Cord 5013-2670

| | | | | | | | PD |
|---|---|---|---|---|---|---|---|
| | | | | Seeded for | | | Time |
| Passage | Total Count | Flow | Cryo | Next Passage | Days | Doubled | (hrs) |
| 7 | 3,420,000 | 3,240,000 | | 180,000 | 4 | | |
| 8 | 1,260,000 | | 1,150,000 | 104,970 | 3 | 7.00 | 10.29 |
| 9 | 841,000 | | | 841,000 | 3 | 8.01 | 8.99 |
| 10 | 9,830,000 | 4,910,000 | 4,740,000 | 175,000 | 4 | 11.69 | 8.21 |
| 11 | 1,976,000 | | | 1,976,000 | 4 | 11.26 | 8.53 |
| 12 | 5,720,000 | | 4,420,000 | 1,300,000 | 2 | 2.89 | 16.58 |
| 13 | 12,110,000 | 4,540,000 | 7,190,000 | 378,000 | 4 | 9.32 | 10.31 |
| 14 | 4,349,000 | | 3,624,000 | 725,000 | 5 | 11.49 | 10.44 |

Phenotype Analysis of Examples 6 and 7

3,240,000 cells comprising the mixed cultures of M2-048, Cord 5006-2180, and Cord 5013-2670 collected at Passage 7 and M2-048 menstrual cell culture were subjected to phenotype analysis according to the method described in this application. The results of the phenotype analysis are shown on TABLE L.

TABLE L
Flow Cytometry Analysis of Cell Culture

| | M2-048 + | M2-048 + | | | | |
|---|---|---|---|---|---|---|
| | CORD | CORD | | | M2- | |
| | (mixed) | (mixed) | M2-048 | M2-048 | 048 + CORD | |
| | P7 | P11 | P11 | P14 | mixed) P19 | |
| | Date: | Date: | Date: | Date: | Date: Mar. | |
| | Feb. 05, 2008 | Feb. 15, 2008 | Feb. 15, 2008 | Feb. 26, 2008 | 21, 2008 | |
| | % POS | % POS | % POS | % POS | % POS | |
| HLA-I | 100 | 86.6 | 97.1 | 98.5 | 96.6 | HLA-I |
| CD133 | 0 | 0 | 0 | 0 | 0 | CD133 |
| HLA-II | 18 | 70.8 | 0.2 | 0.4 | 0.7 | HLA-II |
| CD9 | 95 | 81.9 | 78.6 | 30.7 | 38.2 | CD9 |
| CD54 | 0 | 0 | 1.5 | 4 | 2.3 | CD54 |
| CD45 | 50 | 23.5 | 2 | 5.2 | 5.8 | CD45 |
| CD10 | 15 | 59 | 31.1 | 27.4 | 15.2 | CD10 |
| CD59 | 100 | 82.9 | 95 | 99.1 | 98.3 | CD59 |
| CD63 | 100 | 7.7 | 13.1 | 8 | 3.8 | CD63 |
| CD34 | 0 | 85.3 | 12.1 | 5.5 | 20.3 | CD34 |
| CD13 | 83 | 96.3 | 98.5 | 98.3 | 95.2 | CD13 |
| CD49e | 88 | 86.5 | 94.4 | 90.4 | 69 | CD49e |
| CD49f | 70 | 93 | 97.5 | 96 | 80.6 | CD49f |
| CD81 | 100 | 91.4 | 96.3 | 94.1 | 93.8 | CD81 |
| CD44 | 100 | 86.4 | 93.2 | 99.4 | 99 | CD44 |
| CD117 | 0 | 0 | 0 | 0 | 0 | CD117 |
| CD38 | 0 | 0 | 0 | 0 | 0 | CD38 |
| CD29 | 100 | 98.4 | 95 | 99.4 | 98.1 | CD29 |
| CD105 | 98 | 91.8 | 96.9 | 98.6 | 96.2 | CD105 |
| CD90 | 98 | 93.1 | 95.6 | 94.1 | 92.4 | CD90 |
| CD166 | 99 | 91.7 | 97.3 | 98.8 | 98.8 | CD166 |
| NANOG | ND | 1 | 0.1 | ND | 0.6 | NANOG |
| SSEA3 | 0 | 0 | 0 | ND | 0 | SSEA3 |
| SSEA4 | 0 | 43.9 | 44.9 | ND | 2.6 | SSEA4 |
| CD3 | ND | 0 | 0 | 0 | 0 | CD3 |
| CD19 | ND | 0 | 0 | 0 | 0 | CD19 |
| CD14 | ND | 0 | 0 | 0 | 0.2 | CD14 |
| CD56 | ND | 0 | 0 | 0 | 0 | CD56 |
| CD41 | ND | 85 | 97.8 | 98.7 | 95.6 | CD41 |

### EXAMPLE 8

### Culture of Various Concentrations of M2-048 Menstrual Cells, 5006-2180 Cord Cells, and 5013-2670 Cells

Seven 3 ml tubes of culture media (MethoCult #4034—semi-solid media) were thawed at room temperature and then placed on ice for 15 minutes along with the cell dilutions (25,000 cells of 5006-2180 Cord Cells/ml; 25,000 cells of 5013-2670 Cord Cells/ml; 50,000 cells of 5013-2670 Cord Cells/ml; 1,000 cells of M2-048/ml; 25,000 Cells of M2-048+1,000 cells of 5006-2180 Cord Cells/ml; 25,000 cells of M2-048+1,000 cells of 5013-2670 Cord Cells/ml; and 50,000 cells of M2-048+1,000 cells of 5013-2670 Cord Cells/ml). After 15 minutes, each cell dilution was inoculated into separate tubes of Methocult #4034 semi-solid media. The tubes were gently vortexed and then incubated on ice for 30 minutes. After incubation, the culture media and menstrual cells, cord cells, and menstrual and cord cell combinations were each evenly divided into the wells of seven different 4-well plates for incubation. One ml of DPBS was added to each fourth well of the seven different 4-well plates to assist with humidity, and the cells were incubated at 37° C.

TABLE M
Plate Culture of Cord 5006-2180 Cells, Cord 5013-2670 Cells, M2-
048 Menstrual Cells, M2-048 Menstrual Cells + Cord 5006-2180 Cells,
M2-048 Menstrual Cells + Cord 5013-2670 Cells, and M2-048
Menstrual Cells + Cord 5013-2670 Cells

| | Cord cells per | | | | |
|---|---|---|---|---|---|
| | well/M2 | | | CFU- | |
| | cells per | BFU-E | CFU-GM | GEMM | Days in |
| Sample ID | well | Avg | Avg | Avg | culture |
| 5006-2180 | 25,000 | 52.7 | 14.3 | 0 | 16 |
| 5013-2670 | 25,000 | 0.3 | 1.6 | 0.6 | 16 |
| 5013-2670 | 50,000 | 1.6 | 0.3 | 13 | 16 |
| M2-048 P4 | 1,000 | 0 | 0 | 0 | 23 |
| M2-048 P4 & | 25,000/1,000 | 27 | 11.3 | 2 | 16 |
| 5006-2180 | | | | | |
| M2-048 P4 & | 25,000/1,000 | 5.6 | 4.3 | 1.6 | 16 |
| 5013-2670 | | | | | |
| M2-048 P4 & | 50,000/1,000 | 6.3 | 3 | 1.6 | 16 |
| 5013-2670 | | | | | |

While preferred embodiments of the present invention have been shown and described, it will be apparent to those skilled in the art that many changes and modifications may be made without departing from the invention in its broader aspects. The appended claims are intended to cover, therefore, all such changes and modifications as fall within the true spirit and scope of the invention.

We claim:

1. A population of human cells expressing CD34 obtained from expansion of human cord blood cells in suitable culture conditions with human menstrual cells to promote population doublings of the human cord blood cells.

2. The population of human cells of claim 1, wherein the population of human cells also express SSEA4.

4. The population of human cells of claim 1, wherein the population of human cells also express HLA-II.

5. The population of human cells of claim 1, wherein the population of human cells expressing CD34 are suspended in any one of a cryopreservation agent, a culture media, a growth media, or a differentiation media.

6. The population of human cells of claim 1, wherein the population of human cells expressing CD34 are the result of at least two or more population doublings.

7. The population of human cells of claim 1, wherein the population of human cells are capable of giving rise to any one of colony forming units, colony forming unit granulocyte (CFU-GM) macrophages, burst forming unit erythroids (BFU-E), and colony forming unit granulocyte erythrocyte macrophage megakaryocyte blood lineage precursor cells (CFU-GEMM).

8. A population of human cord blood cells expressing CD34 obtained by the process comprising:
co-culturing a sufficient amount of cord blood stem cells with a sufficient amount of menstrual stem cells in conditions suitable for expansion of the cord blood cells; and
proliferating the sufficient amount of cord blood cells in culture through at least two population doublings.

9. The process of claim 8, wherein the process comprises the further step of growing the sufficient amount of cord blood cells in culture to give rise to any one of colony forming units (CFU), colony forming unit granulocyte macrophages (CFU-GM), burst forming unit erythroids (BFU-E), and colony forming unit granulocyte erythrocyte macrophage megakaryocyte blood lineage precursor cells (CFU-GEMM).

10. The process of claim 8, wherein the process comprises the further step of isolating cord blood cells expressing CD34 after proliferating the sufficient amount of cord blood cells in culture.

11. The process of claim 8, wherein the process comprises the further step of cryo-preserving the population of human cord blood cells expressing CD34 after proliferating the sufficient amount of cord blood cells in culture.

12. The process of claim 8, wherein the population of human cord blood cells expressing CD34 also express HLA-II after proliferating the sufficient amount of cord blood cells under suitable culture conditions.

13. The process of claim 8, wherein the population of human cord blood cells expressing CD34 also express SSEA4 after proliferating the sufficient amount of cord blood cells under suitable culture conditions.

14. The process of claim 8, wherein the proliferating the sufficient amount of cord blood cells in culture comprises growing the cord blood cells to give rise to any one of colony forming units, colony forming unit granulocyte macrophages (CFU-GM), burst forming unit erythroids (BFU-E), and colony forming unit granulocyte erythrocyte macrophage megakaryocyte blood lineage precursor cells (CFU-GEMM).

15. A method for obtaining expanded human cord blood cells expressing CD34, the method comprising:
seeding a sufficient amount of cord blood cells with a sufficient amount of menstrual cells under co-culture conditions suitable to promote expansion of the cord blood cells; and
co-culturing the cord blood cells with the menstrual cells under culture conditions that support at least two or more population doublings of the cord blood cells.

16. The method of claim 15, wherein the co-culturing of the human cord blood cells expressing CD34 comprises expanding cord blood cells to express at least one or more of SSEA-4 and HLA-II.

17. The method of claim 16, wherein the expanded human cord blood cells express high levels of CD34.

18. The method of claim 15, wherein the co-culturing of the cord blood cells comprises expanding cord blood cells to give rise to any one of colony forming units, colony forming unit granulocyte macrophages (CFU-GM), burst forming unit erythroids (BFU-E), and colony forming unit granulocyte erythrocyte macrophage megakaryocyte blood lineage precursor cells (CFU-GEMM).

19. The method of claim 15, wherein the method comprises at least one of the further steps of immunoselecting expanded human cord blood cells for CD34, isolating expanded human cord blood cells firom a culture for infusion into a human, cryopreserving expanded human cord blood cells, or differentiating expanded cord blood cells into a cell lineage.

20. The method of claim 15, wherein the method comprises the step of growing expanded human cord blood cells to give rise to any one of colony forming units, colony forming unit granulocyte macrophages (CFU-GM), burst forming unit erythroids (BFU-E), and colony forming unit granulocyte erythrocyte macrophage megakaryocyte blood lineage precursor cells (CFU-GEMM).
